(19) United States
(12) Patent Application Publication
(10) Pub. No.: US 2009/0316139 A1
(43) Pub. Date: Dec. 24, 2009

(54) BIODIESEL/DIESEL BLEND LEVEL DETECTION USING ABSORBANCE

(76) Inventors: Dev Sagar Shrestha, Moscow, ID (US); Zawadzki Artur, Moscow, ID (US)

Correspondence Address: KLARQUIST SPARKMAN, LLP, 121 SW SALMON STREET, SUITE 1600, PORTLAND, OR, 97204, US

(21) Appl. No.: 12/441,988

(22) PCT Filed: Oct. 11, 2007
(86) PCT No.: PCT/US07/21919

§ 371 Date: Mar. 19, 2009

Related U.S. Application Data
(60) Provisional application No. 60/851,443, filed on Oct. 12, 2006.

(51) Int. Cl. G01N 21/00; G01N 33/22

(52) U.S. Cl. 356/51; 356/437

(57) ABSTRACT

Described herein are embodiments of a method and device for determining the relative amounts of biodiesel and diesel in a biodiesel/diesel blend without separating the biodiesel from the diesel. Embodiments of the method comprise providing a device for measuring absorbance of the blend, measuring absorbance of the blend at one or more wavelengths, and determining the relative amounts of biodiesel and diesel in the blend from the absorbance. Embodiments of the device comprise a light source and a detector for detecting light transmitted through a sample of a biodiesel/diesel blend. Typically the device includes a data analyzer for computing relative amounts of biodiesel and diesel in the blend. Some embodiments of the device further comprise one or more filters, which allow only light of a particular wavelength or wavelengths to pass through the filter.

### CROSS REFERENCE TO RELATED APPLICATION

This application claims the benefit of the earlier filing date of U.S. provisional application No. 60/851,443, filed Oct. 12, 2006, which is incorporated in its entirety herein by reference.

### FIELD

The disclosure pertains to embodiments of a method and device for determining the relative amounts of biodiesel and diesel in a biodiesel/diesel blend.

### BACKGROUND

Biodiesel is defined by the National Biodiesel Board as a fuel comprising mono-alkyl esters of long chain fatty acids derived from vegetable oils or animal fats. Fatty acids in oils and fats are present as triglycerides. Biodiesel is produced by transesterification of the triglycerides with alcohols, most commonly methanol or ethanol, in the presence of a catalyst. The resulting biodiesel is a mixture of fatty acid esters. The types and relative amounts of fatty acid esters in the mixture depend on the feedstock used.

Biodiesel contains essentially no aromatic compounds. Aromatic compounds are a large class of unsaturated cyclic hydrocarbons containing one or more rings. Benzene is a typical aromatic compound, which has a 6-carbon ring containing three double bonds. Certain 5-membered cyclic compounds, such as the furan group, also are aromatic compounds.

Diesel fuel is distilled from crude petroleum, comprising primarily aliphatic alkanes (paraffins), cycloalkanes (naphthenes), and aromatic hydrocarbons. One feature of diesel fuel is the presence of about 20%-35% aromatic compounds by weight.

Biodiesel and diesel have many common characteristics. Biodiesel is suitable for use in diesel engines without any engine modification. However, there are some important differences between the two fuels. Because of these differences, many engine manufacturers recommend limiting the amount of biodiesel blended with diesel fuel.

The blend level (biodiesel percentage in a biodiesel-diesel mixture) determines many important characteristics of the blended fuel. Blends of biodiesel and diesel are designated by the letter “B” and a number denoting the biodiesel percentage within the blend, i.e., B5, B10, etc. Using biodiesel/diesel blends having higher biodiesel levels than recommended may compromise engine performance. Lower blend levels may reduce expected benefits such as fuel lubricity and lower tail pipe emissions of unburned hydrocarbons, carbon monoxide, particulate matter, nitrogen oxides, sulfates, polycyclic aromatic hydrocarbons (PAHs), and nitrated PAHs. In addition, biodiesel cloud and pour points are usually higher than those of diesel fuel. The cloud point is the temperature at which the fuel becomes hazy or cloudy due to wax crystal formation. The pour point is the lowest temperature at which an oil will flow. As a result, as the percentage of biodiesel increases (i.e., a higher blend level) the fuel blend becomes unsuitable or difficult to use in cold weather conditions. Further, engine injection timing can be adjusted based on the blend level in order to improve the engine emission and performance.

Actual biodiesel content in fuel sold at gas stations may be significantly different from the stated blend level. There are several reasons why the actual blend level may differ from the stated level. For instance, if biodiesel is blended at a temperature less than 10° F. above its cloud point, it does not mix well with diesel. This may cause a rich mixture in one portion of a tank versus a lean mixture in another portion (National Biodiesel Board, 2005). Other reasons may include profit-driven fraud and mixing additional diesel into the blend. Biodiesel is usually sold at a higher price than diesel fuel; therefore, the price of a fuel blend depends on the blend level.

Although prior methods and devices have been utilized to detect biodiesel blend levels, these methods and devices have significant limitations and/or cost disadvantages. Knothe (JAOCS, Vol. 78, No. 10, pp. 1025-1028, 2001) has shown that Near Infrared (NIR) spectroscopy and Nuclear Magnetic Resonance (NMR) can be used to determine relative amounts of biodiesel and diesel in blends. However, the NMR method depends on the biodiesel fatty acid profile, which varies based on the biodiesel feedstock. In addition, using NMR to detect the blend level is prohibitively expensive. Additionally, NMR cannot readily be implemented at point-of-sale locations.

For NIR spectroscopy, Knothe suggested using wavelength(s) around 1665 nm or 2083-2174 nm. Knothe, however, tested only a single source of soy methyl ester. This small sample size provides no general information as to the suitability of IR for determining blend levels. Further, since aromatic compounds produce infrared bands due to the relatively rigid molecular structure (Workman, Handbook of Organic Compounds, 2001) and diesel fuels have varying amounts of aromatics, IR absorbance of a blend may not directly correlate to the biodiesel percentage alone. Thus, calibration is usually needed when using IR to determine blend levels. Further IR spectroscopy is generally more expensive to use than ultraviolet spectroscopy and is somewhat temperature dependent.

Pimentel et al. (Microchemical Journal, Vol. 82, Issue 2, pp. 201-206, 2006) used middle- and near-infrared spectroscopy to determine biodiesel content blended with mineral diesel fuel. The results showed infrared spectra, to be “ . . . suitable as practical analytical methods for predicting biodiesel content in conventional diesel blends in the volume fraction range from 0% to 5%.” (Abstract, 11. 4-6) However, this range is too narrow to be suitable for commercial blends that range up to 80% (v/v) biodiesel.

Tat and Van Gerpen (Applied Engineering in Agriculture, 19(2), pp. 125-131, 2003) used a commercially available dielectric fuel composition detector to determine biodiesel blend level. The authors concluded in a related earlier paper that “[T]he error was about 10.5 percent.” (ASAE Meeting Presentation, Paper No. 01-6052, 2001.) This level of accuracy, however, may not always be suitable for biodiesel blend level determinations.

Ritz and Croudace (Petrospec Application Note, Petroleum Analyzer Company, L.P., 2005) disclose using a diesel fuel analyzer “CETANE 2000,” a commercial apparatus capable of measuring “ . . . density, cetane number and cetane index plus cetane improver, total aromatics, polynuclear aromatic and biodiesel content in one portable instrument.” (p. 2.) The instrument uses infrared (IR) absorbance at 5731 nm (1745 cm⁻¹) and 8621 nm (1160 cm⁻¹) to target the C—O stretch in the biodiesel fatty acid esters. Since CETANE 2000 is designed to detect several fuel parameters simultaneously, for a blend level detection application, it may not be a cost effective solution.

Foglia et al. (Chromatographia, 62(3/4): 115-119, 2005) disclose using high performance liquid chromatography (HPLC) to quantify biodiesel blends. “Separated components were quantitated using either an evaporative light scattering detector (ELSD) or UV detector.” (Technical abstract, 11. 4-5.) The primary disadvantage to this method is the requirement to first separate the components within the blend before quantitation.

Despite the above methods for quantifying biodiesel blends, there still exists a need for an inexpensive method and device that do not require separating the biodiesel and diesel components and that can be optionally used in the field.

### SUMMARY

Described herein are embodiments of a method and a device for determining the relative amounts of biodiesel and diesel in a biodiesel/diesel blend without separating the biodiesel from the diesel. Embodiments of the method comprise providing a device for measuring absorbance of the blend, measuring absorbance of the blend at one or more wavelengths, and determining the relative amounts of biodiesel and diesel in the blend from the absorbance.

In some embodiments of the method, a biodiesel/diesel blend is obtained. In other embodiments, biodiesel is produced and mixed with diesel fuel to form a blend. In some embodiments of the method, the blend is diluted with a non-aromatic solvent. The absorbance of the diluted blend is subsequently measured at one or more wavelengths. In some embodiments, the wavelengths are within the near ultraviolet range, typically in the range from about 200 nm to about 320 nm. For example, absorbance may be measured at plural wavelengths in the range from about 250 nm to about 300 nm. In some embodiments, the wavelengths are within the near infrared (NIR) range, typically in the range from about 750 nm to about 1100 nm. For example, the absorbance may be measured at plural wavelengths in the range from about 750 nm to about 1000 nm. In some embodiments, absorbance is measured at wavelengths both within the near ultraviolet range and within the near infrared range. The absorbance measurements are used to determine the relative amounts of biodiesel and diesel in the blend.

Embodiments of the device useful for determining biodiesel/diesel blend proportions comprise a light source and a detector for detecting light transmitted through a sample of a biodiesel/diesel blend. Typically the device includes a data analyzer for computing relative amounts of biodiesel and diesel in the blend, although this computation can also be done manually.

In some embodiments of the device, one or more filters are effectively coupled to a disk. Each filter allows only light of a particular wavelength or wavelengths to pass through the filter. The disk is located between the light source and the detector. The disk is operably coupled to a motor. The motor moves the disk to align one of the filters between the light source and the detector. For example, the motor may rotate the disk through a predetermined angle of rotation at a set time interval to align one of the filters between the light source and the detector. The detector outputs a voltage signal to a data analyzer where the signal is proportional to the intensity of light striking the detector.

In some embodiments of the device, three filters are used. The filters are effectively coupled to a disk. The motor rotates the disk with the filters such that each of the filters is aligned in turn between the sample and the detector. As each filter is aligned, the detector outputs a voltage signal proportional to the intensity of light detected by the detector to the data analyzer. In some embodiments, the detector outputs a plurality of voltage signals as each filter is aligned. The data analyzer correlates each signal to an absorbance measurement. The data analyzer further computes an absorbance index from the absorbance measurements and determines the blend level from the absorbance index.

The foregoing and other objects, features, and advantages of the disclosed method and apparatus will become more apparent from the following detailed description, which proceeds with reference to the accompanying figures.

### BRIEF DESCRIPTION OF THE DRAWINGS

FIG. 1 is a block diagram of an embodiment of a method for determining the percentage of diesel in a biodiesel/diesel blend.

FIG. 2 is a block diagram of an embodiment of a method for determining the percentage of diesel in a biodiesel/diesel blend.

FIG. 3 is a block diagram of an embodiment of a method for determining the percentage of diesel in a biodiesel/diesel blend.

FIG. 4 is a series of absorbance curves for various blends of soy methyl esters and No. 2 diesel.

FIG. 5 is a graph of percent biodiesel in blends versus absorbance.

FIG. 6 is a graph of coefficients of determination plotted against wavelength.

FIG. 7 is a series of absorbance spectra for diesel fuels.

FIG. 8 is a series of absorbance curves for various blends of mustard methyl esters and diesel fuel.

FIG. 9 is a graph of biodiesel blend level versus the ratio of diesel absorbance to biodiesel absorbance.

FIG. 10 is a front perspective view of one embodiment of a device useful for determining relative amounts of biodiesel and diesel in a biodiesel/diesel blend.

FIG. 11 is a graph of absorbance index versus percent biodiesel.

FIG. 12 is a graph of biodiesel blend level versus the ratio of diesel absorbance to biodiesel absorbance for various sources of biodiesel and diesel.

FIG. 13 is a flow chart of the software instructions performed by one embodiment of a data analyzer.

### DETAILED DESCRIPTION

The presence of aromatics in diesel and their absence in biodiesel provides a basis for distinguishing these two fuels in a blend using near ultraviolet spectroscopy. This can be accomplished without separating the blend components. Benzene, a simple aromatic compound present in diesel, has maximum absorption at 278 nm (Bruno and Svoronos, Handbook of basic tables for chemical analysis, 2^{nd} ed., 2003). Biodiesel has negligible absorbance at the same frequency. Hence, biodiesel produced from different feedstocks will not interfere with absorbance measurements at this wavelength. Near ultraviolet (near UV) comprises electromagnetic radiation from about 200 nm to about 380 nm.

Differences in the near infrared (near IR) spectra of biodiesel and diesel also provide a basis for distinguishing the two fuels in a blend without separating the components. Near IR comprises electromagnetic radiation from about 750 nm to about 2500 nm.

In general, light sources, filters and detectors are less expensive in these wavelength ranges than using IR or NMR spectroscopy. Thus, near UV and near IR spectroscopy provide a low cost, alternative method for biodiesel blend level determination without separating the blend components.

Embodiments of a method and a device for determining the relative amounts of biodiesel and diesel in a biodiesel/diesel blend using spectroscopy are described below. The method and device provide advantages over the methods and devices described in the prior art. Specifically, the method and device provide a portable, relatively low cost and accurate determination of biodiesel blend levels in the field. For example, a refinery might use the disclosed method and device to confirm that a biodiesel/diesel blend produced has the desired composition. In another example, an outlet, such as a gas station, might use the disclosed method and device to verify the composition of a delivered biodiesel/diesel blend.

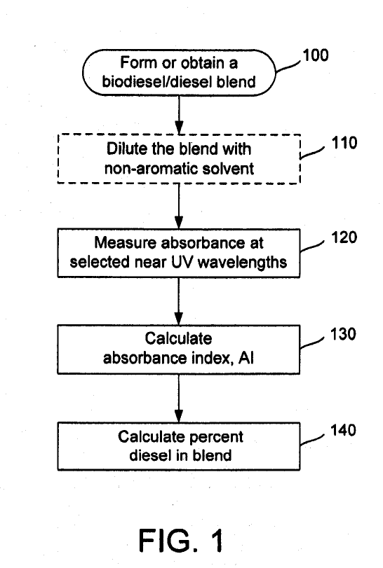

FIG. 1 discloses one embodiment of the method. A biodiesel/diesel blend 100 is produced or obtained. The blend is typically diluted with a suitable solvent in step 110. Suitable diluents include solvents that are non-aromatic and are miscible with the blend. Preferred diluents include lower alkyl alkanes (typically defined as containing 1-10 carbons) and combinations thereof. The working embodiments described herein used n-heptane as a diluent.

Alternatively, the blend is not diluted. For example, signal processing techniques could adjust signal strength output from a spectroscopic device and eliminate the need for dilution.

Light is passed through an aliquot of blend 100, which is typically diluted, and light absorbance is measured in step 120. Preferably, the absorbance is measured at different wavelengths within the near UV range from about 250 nm to about 300 nm. In general, the more wavelengths sampled, the greater the accuracy of the blend percent determination. In a working embodiment, three wavelengths in the range from about 260 nm to about 280 nm provided suitable data. However, more than three wavelengths can be used. For example, plural wavelengths, including at particular intervals, such as at 5-nm wavelength intervals, could be used. Utilizing the absorbance values, an absorbance index (AI) is calculated in step 130. The absorbance index is proportional to the amount of diesel in the blend 100. Recognition of this correlation, and determination of the absorbance, allows calculation of the percent diesel in the blend in step 140.

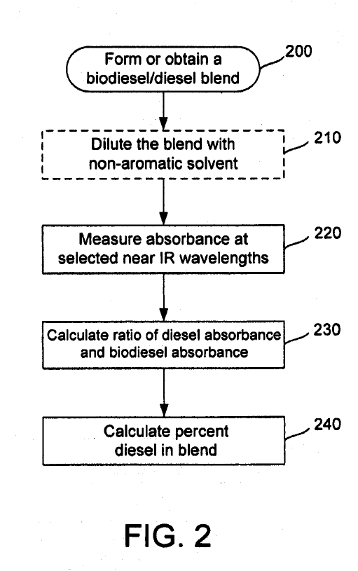

FIG. 2 discloses another embodiment of the method. A biodiesel/diesel blend 200 is produced or obtained. The blend is typically diluted with a suitable solvent in step 210. Suitable diluents include solvents that are non-aromatic and are miscible with the blend. Preferred diluents include lower alkyl alkanes (typically defined as containing 1-10 carbons) and combinations thereof. Alternatively, the blend is not diluted.

Light is passed through an aliquot of blend 200, and light absorbance is measured in step 220. Preferably, the absorbance is measured at different wavelengths within the near IR range from about 750 nm to about 1100 nm. In general, the more wavelengths sampled, the greater the accuracy of the blend percent determination. In a working embodiment, four wavelengths in the range from about 750 nm to about 1000 nm provided suitable data. Utilizing the absorbance values, a ratio of diesel absorbance to biodiesel absorbance is calculated in step 230. The ratio is proportional to the amount of diesel in the blend 200. Recognition of this correlation, and determination of the absorbance, allows calculation of the percent diesel in the blend in step 240.

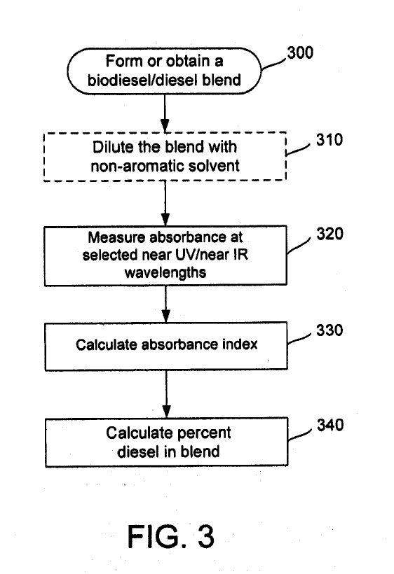

FIG. 3 discloses another embodiment of the method. A biodiesel/diesel blend 300 is produced or obtained. The blend is typically diluted with a suitable solvent in step 310. Suitable diluents include solvents that are non-aromatic and are miscible with the blend. Preferred diluents include lower alkyl alkanes (typically defined as containing 1-10 carbons) and combinations thereof. Alternatively, the blend is not diluted.

Light is passed through an aliquot of blend 300, and light absorbance is measured in step 320. Preferably, the absorbance is measured at different wavelengths, where at least one wavelength is within the near UV range from about 250 nm to about 300 nm and at least one wavelength is within the near IR range from about 750 nm to about 1100 nm. In one embodiment, absorbance is measured at a plurality of wavelengths within the range from about 250 nm to about 300 nm and one wavelength within the range from about 750 nm to about 1100 nm. Utilizing the absorbance values, an absorbance index is calculated in step 330. The absorbance index is proportional to the amount of diesel in the blend 300. Recognition of this correlation, and determination of the absorbance, allows calculation of the percent diesel in the blend in step 340.

### Production of Biodiesel Blends

Biodiesel is produced from vegetable oils and/or animal fats. Vegetable oils and animal fats comprise triglycerides, having three fatty acids bonded as esters to glycerol. Fatty acids are long-chain carboxylic acids, i.e., with a carbon chain up to about 30 carbon atoms in length, more typically from about 4 to about 24 carbon atoms in length.

To produce biodiesel, the fatty acids must be cleaved from the glycerol. Transesterification is a process in which the fatty acid chains are cleaved from the glycerol and converted to fatty acid esters. Transesterification comprises reacting the triglycerides with an alcohol in the presence of a catalyst. Suitable alcohols include primary lower alkyl alcohols. Typical primary alcohols used in transesterification include methanol or ethanol. Suitable catalysts include bases, acids, amines, metal oxides, metal alkoxides, among others. Some examples of suitable catalysts are sodium hydroxide, potassium hydroxide, sodium methoxide, sodium silicate, sulfuric acid, hydrochloric acid, sulfonic acid, sodium methylate, and potassium methylate. Commercially, base catalysts are preferred due to the low temperature and pressure requirements for the transesterification reaction and high conversion to transesterified products of about 98% with minimal side reactions and time. More preferably, the base catalyst is a Group I hydroxide, such as sodium hydroxide or potassium hydroxide.

After transesterification, the two major products are glycerol and fatty acid esters (biodiesel). The products are immiscible and are separated by any suitable method, including gravity or centrifugation. Excess unreacted alcohol is removed from both the biodiesel and glycerol. Preferably, the alcohol is removed by distillation or flash evaporation. The recovered alcohol can be reused. In some embodiments, the biodiesel is used without further purification. In other embodiments, the biodiesel is further purified by washing with water. The water is subsequently removed. In other embodiments, the biodiesel is distilled to remove colored components and produce a colorless biodiesel. The biodiesel is then mixed with diesel to produce the desired blend(s).

### Near Ultraviolet Spectroscopy of Biodiesel/Diesel Blends

Biodiesel/diesel blends have very high absorbance in the UV range. As one of ordinary skill in the art readily appreciates, errors in absorbance measurement are lowest when the absorbance value is below 2. Neat blends of biodiesel/diesel typically have an absorbance of 2. In order to bring the absorbance of the blends within the measurable range of a spectrophotometer, blends typically are diluted with a solvent. Suitable diluents include solvents that are non-aromatic, do not absorb light in the utilized wavelength range, and are miscible with the blend. Preferred diluents include lower alkyl alkanes and combinations thereof. The embodiments described herein used n-heptane as a diluent. Alternatively, signal processing techniques are used to obtain an absorbance measurement without diluting the blend.

The blends are diluted using standard volumetric techniques to a concentration that produces an absorbance value of about 2 or less. For working embodiments, the blends were diluted to a final concentration from about 0.02% to about 0.05% (v/v). Typically the blends are diluted to a final concentration from about 0.03% to about 0.04% (v/v). In some embodiments, the blends are diluted in successive steps to ensure accuracy. In a working embodiment of the method, the blends were diluted to a final concentration of 0.034% (v/v) with n-heptane in three successive steps.

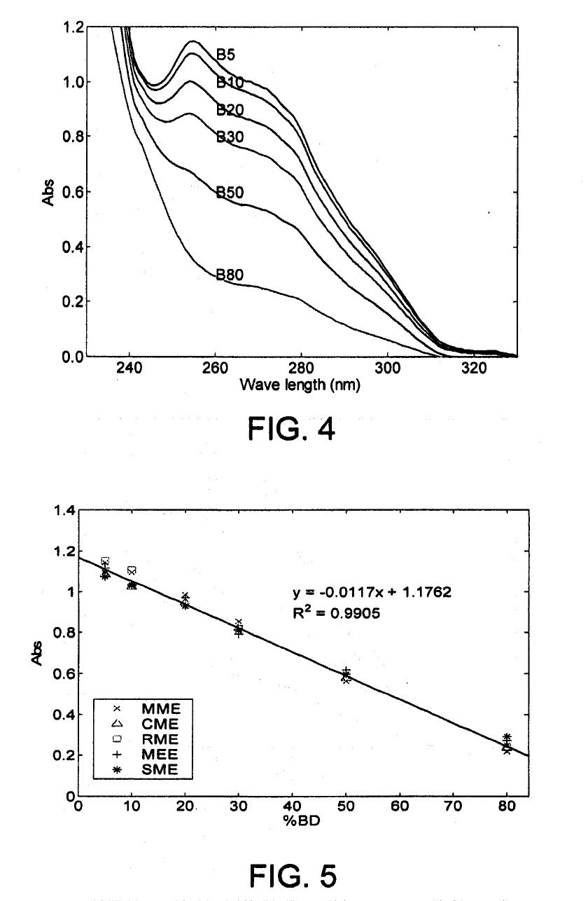

In a working embodiment, the UV absorption spectra of biodiesel samples and biodiesel blends with diesel were measured using a Beckman Coulter® DU520 single-beam general purpose spectrophotometer (Fullerton, Calif.). Diluted samples of different biodiesel/diesel blends were placed in standard 1-cm quartz cuvettes, and absorption spectra in the range of 190-350 nm at 1-nm intervals were determined. FIG. 4 shows a typical series of absorbance curves for diluted soy methyl esters in the range from 240 nm to 320 nm. The soy methyl esters blended with No. 2 diesel were diluted 1:2915 in n-heptane.

When percent biodiesel was plotted against absorbance for any given wavelength between 245-305 nm, a linear relationship was observed. The absorbance decreased linearly with the increasing blend level. This decrease was attributed to the decreasing concentration of aromatic compounds present in the diesel. The difference in absorbance for the B5 and B80 blends was highest from about 255 nm to about 265 nm.

FIG. 5 is a graph of absorbance at 260 nm versus percent biodiesel. Biodiesel/diesel blends were formed from different feedstocks and diluted. In the graph legend, the abbreviations are as follows: MME—mustard ethyl esters, CME—canola methyl esters, RME—rapeseed methyl esters, MEE—mustard ethyl esters, and SME—soybean methyl esters. For all measured feedstocks, the graph shows a linear relationship with a high coefficient of determination (R²=0.9905).

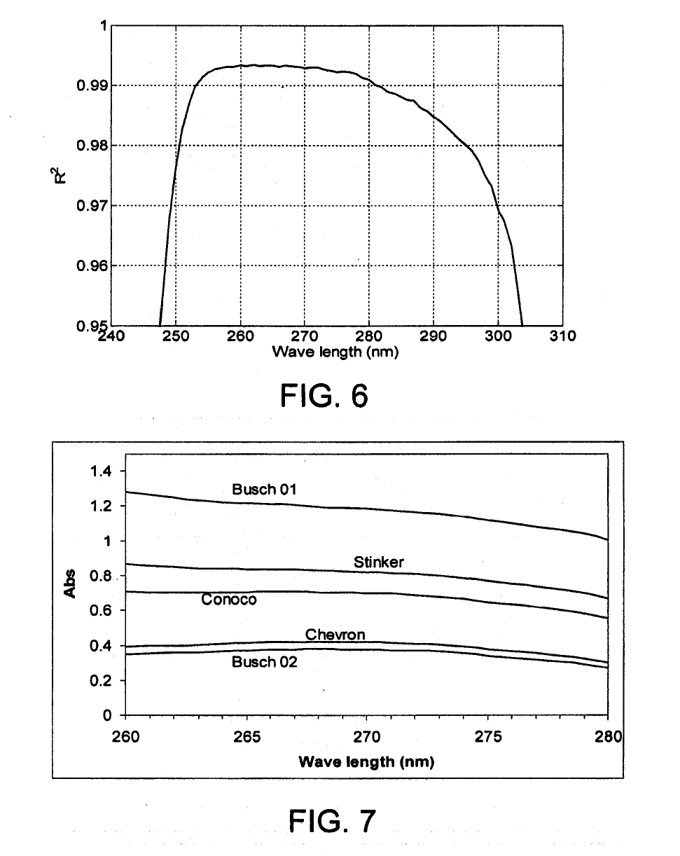

In order to find the wavelength for the best correlation, R² was calculated for each wavelength from 245-305 nm and plotted against the corresponding wavelengths as shown in FIG. 6. The R² value was greater than 0.99 for the wavelengths from 254 nm to 281 nm and dropped sharply outside of this range.

The highest R² of 0.9933 was observed at 263 nm. Based on this analysis, it was concluded that a single wavelength between 250 nm and 300 nm could be used to detect the blend level regardless of biodiesel feedstock. In field applications, however, it is expected that the aromatic content will vary from one diesel fuel to another. Thus, a single wavelength used as the absorbance magnitude varies based on the diesel fuel's aromatic content.

An absorbance data transformation procedure, as discussed below, was developed to eliminate the differences in the absorbance intensity coming from various diesel fuels. As mentioned earlier, the aromatic content of diesel varies from about 20% to about 35%. Diesel fuels were collected locally from various gas stations at various times of the year. The diesel fuels were diluted in n-heptane and absorbance was measured. FIG. 7 shows the absorbance spectra.

As discussed earlier, the biodiesel and diesel blends showed a linear variation in absorbance from biodiesel to diesel. However, because the aromatic content of diesel fuels varies, the absorbance of the neat diesel must be measured in order to correlate absorbance with blend level using a single measurement.

A chemical component's absorbance is proportional to its concentration in a solution. When the component is diluted, its absorbance at each wavelength decreases proportionately. The spectrum's shape remains the same after dilution, but the amplitude is attenuated. For instance, if the absorbance of diesel at 260 nm and 270 nm were 1 and 2 respectively, the difference in absorbance is 1. When the diesel is mixed in equal proportion to biodiesel, it is expected that the absorbance would be 0.5 and 1 for the same wavelengths. The difference in absorbance is now 0.5. Thus, the difference in amplitude varies proportionately with the percentage of diesel in the sample.

When measuring the differences in amplitude between two or more wavelengths, the various diesel samples should have a similarly shaped absorbance curve. Further, the samples should have the same amplitude difference between selected points when diluted to the same diesel concentration. As seen in FIG. 7, it was observed that the absorbance curves in the 260 nm to 280 nm range were consistently shaped. Therefore, absorbance measurements within that range are utilized to calculate an absorbance index. Absorbance index (AI) measures the shape of the curve. More specifically, AI indicates changes in absorbance amplitude over a chosen wavelength range in which the absorbance curves are similarly shaped. AI is proportional to the diesel percentage in the measured sample. AI is defined as:

$$AI = {10}^{( {A}_{2} - \frac{{A}_{1} + {A}_{3}}{2} )} \qquad ( Eq . 1 )$$

AI is the absorbance index, and A₁, A₂ and A₃ are absorbance measurements at three wavelengths within the chosen range, wherein A₂ is between A₁ and A₃. AI was found to be linearly correlated with the blend level.

### Near Infrared Spectroscopy of Biodiesel/Diesel Blends

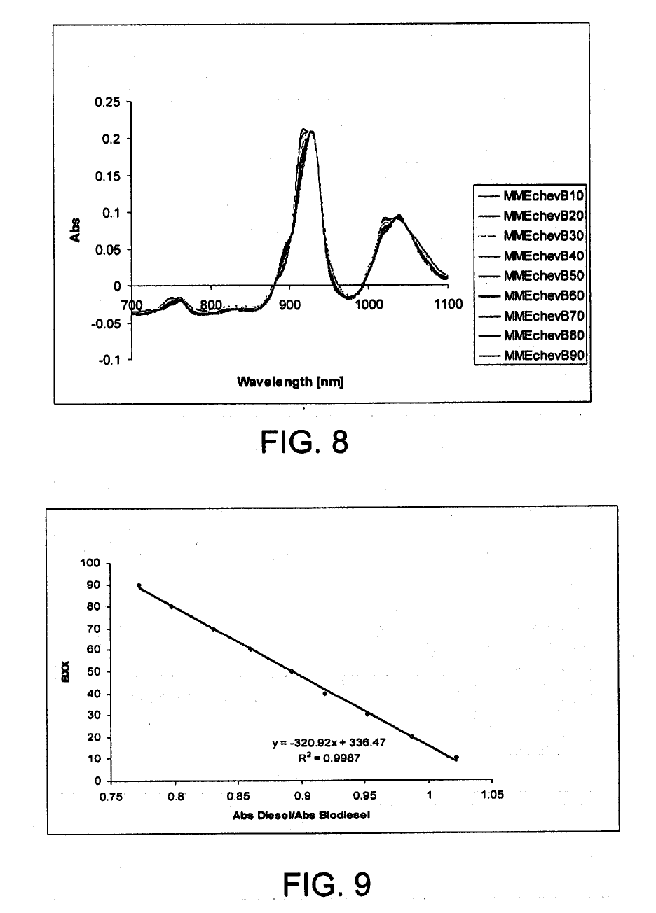

In a working embodiment, biodiesel/diesel blends were prepared and placed into 5-cm cuvets. A Beckman Coulter® DU520 single-beam general purpose spectrophotometer was used to obtain IR spectra of the blends. FIG. 8 is a series of absorbance curves for various blends of mustard methyl esters and diesel fuel. Diesel fuel shows a characteristic absorbance peak at around 917 nm, whereas biodiesel shows a distinctive absorbance peak at around 929 nm. Additionally, there is another characteristic diesel absorption peak at 1020 nm and a biodiesel absorption peak at 1039 nm.

When measuring the absorbance of biodiesel/diesel blends, the absorbance ratio of the two peaks at 917 nm and 929 nm changes. As the percentage biodiesel increases in the blend, the diesel absorbance gradually decreases at 917 nm and the biodiesel absorbance gradually increases at 929 nm. A linear relationship was found between the ratio of these characteristic peaks and the blend level of biodiesel. The ratio is calculated according to Eq. 2 below:

$${Ratio}_{AbsDiesel / AbsBiodiesel} = \frac{{A}_{1} - \frac{{A}_{3} + {A}_{4}}{2}}{{A}_{2} - \frac{{A}_{3} + {A}_{4}}{2}} \qquad ( Eq . 2 )$$

A₁, A₂, A₃ and A₄ are absorbance measurements at four wavelengths within the chosen range. In one embodiment, the four wavelengths were 917 nm, 929 nm, 970 nm and 800 nm, respectively. FIG. 9 is a graph of biodiesel blend level (BXX, where XX is the percentage of biodiesel in the blend) versus the ratio of diesel absorbance to biodiesel absorbance.

### Device for Determining Blend Level

A device for determining the relative amounts of biodiesel and diesel in a biodiesel/diesel blend using spectroscopy is described below. One embodiment of the device comprised a light source and a detector for detecting ultraviolet light transmitted through a sample of a biodiesel/diesel blend. The light source comprises any suitable light source capable of producing light within the range of from about 200 nm to about 320 nm. For example, the light source is a light source producing at least ultraviolet light. Alternatively, the light source is capable of emitting only a single wavelength or discrete wavelengths. For example, the light source could be at least one light-emitting diode that produces a discrete wavelength between the range of from about 200 nm to about 320 nm. A plurality of light-emitting diodes also may be used. Suitable light sources can be obtained from Edmund Optics, among others.

Another embodiment of the device comprised a light source and a detector for detecting infrared light transmitted through a sample of a biodiesel/diesel blend. The light source comprises any light source capable of producing light within the range of from about 750 nm to about 1100 nm. For example, the light source could be a light-emitting diode producing at least infrared light. A suitable light source can be obtained from RadioShack, among others.

A sample of a biodiesel/diesel blend is placed between the light source and the detector. The sample is placed into a cuvet. In a working embodiment, a 1-cm quartz cuvet was used. The cuvet is placed into a sample holder. The sample holder is positioned between the light source and the detector. The sample holder contains two apertures located on opposite sides of the sample holder and aligned with one another to allow light from the light source to pass through the sample and be detected by the detector.

Typically the device also comprises a housing. The housing encloses the light source, sample holder and detector. The housing preferably is substantially “light-tight” to preclude or at least substantially preclude ambient light from entering the housing. The housing preferably comprises an opening positioned substantially near the sample holder. The housing further comprises a lid or cover over the opening, which may be opened or removed to place the biodiesel/diesel blend sample into the sample holder. The lid or cover is then closed or replaced prior to proceeding.

In some embodiments, the device further comprises at least one filter. Alternatively, a plurality of filters is used. The filter allows only light of a particular wavelength or wavelengths to pass through the filter. Typically the filter is an interference or bandpass filter, such as those commonly used as wavelength selectors. Suitable filters can be obtained from Edmund Optics, among others.

The filter or filters are effectively coupled to a filter holder. The filter holder is located between the light source and the sample holder. Alternatively, the filter holder is located between the sample holder and the detector. In some embodiments, the filter holder is coupled to a motor. The motor moves the filter holder to align a filter between the light source and the detector. Light produced by the light source passes through the sample and the filter before being detected by the detector.

The detector comprises a sensor that outputs a voltage signal. The voltage signal is proportional to the intensity of the light being detected by the detector. In some embodiments, the detector is coupled to a circuit board. The circuit board is configured to provide basic signal conditioning. In a working embodiment, the circuit board provided signal amplification. The circuit board is coupled to a data analyzer.

The data analyzer is any device capable of correlating the voltage signal output from the detector to absorbance. Absorbance is defined as the logarithm of the reciprocal of transmittance:

$$A = log ( \frac{1}{T} ) where T = \frac{{I}_{t}}{{I}_{0}} \qquad ( Eq . 3 )$$

where A is absorbance, T is transmittance, Iₜ is transmitted light, and I₀ is incident light. The data analyzer could comprise, for example, a computer having data acquisition and analysis software, a stand-alone microprocessor, or other suitable device.

In some embodiments, the data analyzer contains instructions recorded on any suitable media for implementation by the data analyzer. These instructions enable the data analyzer to further calculate absorbance index, AI, as defined in Eq. 1 or the ratio of diesel absorbance to biodiesel absorbance, as defined in Eq. 2. The data analyzer subsequently calculates blend level from AI, or the ratio of diesel absorbance to biodiesel absorbance, as further outlined in the working examples below. Alternatively, a person manually calculates the absorbance index, or ratio of diesel absorbance to biodiesel absorbance, and blend level from the absorbance of the blend.

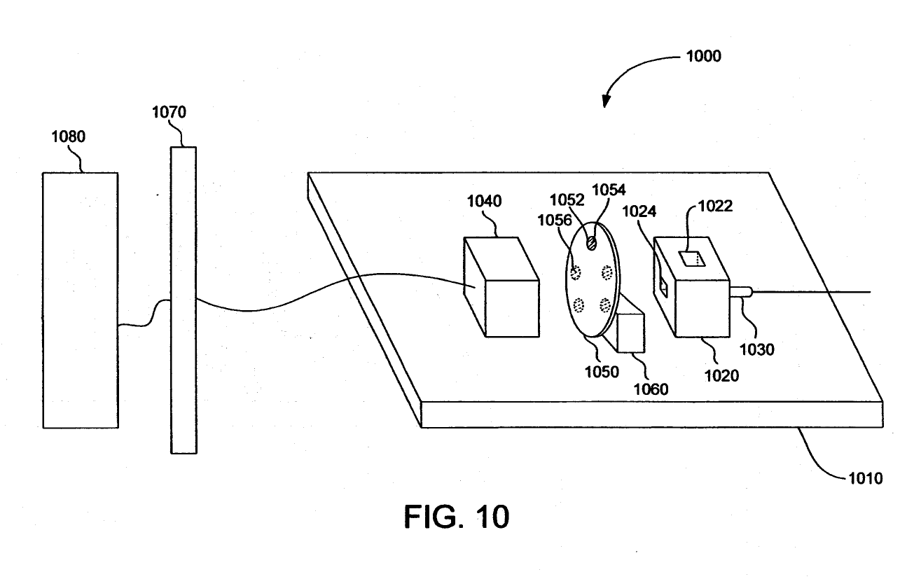

A front perspective view of one working embodiment of the device is shown in FIG. 10. The device 1000 comprises a housing (not shown), a horizontal base 1010, a sample holder 1020, a light source 1030, a detector 1040, a disk 1050 containing one or more filters 1054, 1056, a motor 1060, a circuit board 1070 and a data analyzer 1080.

Sample holder 1020 is mounted on a base 1010. Sample holder 1020 has an aperture 1022 in a substantially central location on the upper surface. The aperture 1022 is cooperatively dimensioned to receive a cuvet, such as a 1-cm cuvet, and extends vertically down into the sample holder 1020. Sample holder 1020 has two apertures 1024 located on opposite sides of the sample holder. The two apertures 1024 are aligned vertically and horizontally with each other and extend horizontally into aperture 1022.

A light source 1030 is effectively positioned, such as being mounted on the base 1010, such that light produced by the light source passes through the two apertures 1024 in the sample holder 1020. In a working embodiment, the light source 1030 was a Spectroline® 40759 short wave UV-C pencil lamp capable of producing ultraviolet light.

A detector 1040 is effectively coupled to the base 1010 such that the sample holder 1020 is located between the detector and the light source 1030. The detector 1040 is mounted such that it aligns with apertures 1024. Light passing through sample holder 1020 is detectable by the detector. The detector 1040 comprises a sensor that outputs a voltage signal. The magnitude of the voltage signal is proportional to the intensity of light striking the detector.

A disk 1050 is operably coupled to a motor 1060 such that the motor rotates the disk. The motor 1060 is capable of rotating the disk 1050 through a predetermined angle of rotation at a set time interval. In one working embodiment, the motor 1060 rotated the disk 1050 through an angle of 90° at one-second intervals. The motor 1060 is mounted to the base 1010 such that the disk 1050 coupled to the motor is aligned between the sample holder 1020 and the detector 1040. Alternatively, the disk 1050 and motor 1060 is aligned between the light source 1030 and the sample holder 1020.

In a working embodiment, the motor 1060 was a Hitec HS 311 servo motor (Hitec RCD®) operably coupled to a computer via a National Instruments 6023E data acquisition board. LabVIEW® software (National Instruments) was utilized to control the motor position.

At least one filter 1054 is mounted into an opening 1052 formed through disk 1050. In some embodiments, additional filters 1056 are mounted into openings 1052 formed through the disk 1050. Typically the filters 1054, 1056 are interference or bandpass filters.

In a working embodiment, three filters 1054 were mounted within disk 1050, the filters being spaced at 90° intervals. The three filters 1054 were selected to allow three different wavelengths within the desired range of 260 nm to 280 nm to pass through to the detector 1060. The filters used were model Nos. 03 FIU 002 (260 nm), 03 FIU 115 (266 nm) and 03 FIM 018 (280 nm) obtained from Melles Griot. A fourth opening 1052 in the disk 1050 did not include a filter and was used to measure light source intensity. The motor 1060 and disk 1050 were adjusted such that the disk rotated 90° at one-second intervals to align one of the openings 1052 between the aperture 1024 and the detector 1040.

In another embodiment, the light source 1030 was an IR LED (RadioShack). A filter 1054 was mounted within disk 1050. The filter was model No. 03 FII 521 (950 nm) obtained from Melles Griot.

A housing (not shown) is mounted to the base 1010 and encloses the sample holder 1020, light source 1030, detector 1040, disk 1050, and motor 1060. The housing is constructed such that ambient light cannot enter the detector 1040. The housing preferably is substantially “light-tight” to preclude or at least substantially preclude ambient light from entering the housing. The housing preferably comprises an opening positioned substantially near the sample holder. The housing further comprises a lid or cover over the opening, which may be opened or removed to place the biodiesel/diesel blend sample into sample holder 1020. The lid or cover is then closed or replaced prior to proceeding.

The detector 1040 comprises a sensor that outputs a voltage signal proportional to the intensity of light striking the detector. The detector 1040 is coupled to a circuit board 1070. The circuit board 1070 is coupled to a data analyzer 1080. The data analyzer 1080 could be any device capable of correlating the voltage signal output from the detector 1060 to absorbance. In a working embodiment, a computer with LabVIEW® graphical programming software was used.

In a working embodiment, the data analyzer 1080 further calculated absorbance index, AI, as defined in Eq. 1. Instructions for performing the data analysis are recorded on any suitable media for implementation by data analyzer 1080. Thus, data analyzer 1080 also is utilized to subsequently calculate blend level from AI as further outlined in the working examples below. Alternatively, the data analyzer 1080 provides the absorbance values, with the investigator subsequently completing the calculations of absorbance index and blend level.

In view of the many possible embodiments to which the principles of the disclosed invention may be applied, it should be recognized that the embodiments described below are provided to illustrate certain features of working embodiments of the invention. These embodiments are only preferred examples of the invention and should not be taken as limiting the scope of the invention.

### EXAMPLES

### Production of Biodiesel and Biodiesel/Diesel Blends

Biodiesel was made from six different feedstocks at the Biological and Agricultural Engineering Laboratory at the University of Idaho. The six feedstocks chosen were canola, soybean, rapeseed and three different mustard cultivars. Seeds were crushed for oil using a mechanical oil expeller. Biodiesel was produced by transesterification of the triglycerides in the oils with primary alcohols in the presence of a catalyst, as is well known in the art. The resulting biodiesel was washed with water as needed to meet industry specifications (ASTM D6751). In working embodiments, the catalyst was sodium methoxide. In one embodiment, methanol was used to produce methyl esters. In another embodiment, one mustard variety was transesterified with ethanol to test the method with ethyl esters.

To produce the biodiesel/diesel blends, five samples of diesel were collected from different gas stations at different times since the amount of aromatic compounds in diesel may vary with the source and the date sampled. Biodiesel/diesel blends containing 5-80% (v/v) biodiesel were prepared using standard volumetric techniques.

### Dilution of Biodiesel/Diesel Blends

In one embodiment of the method, the biodiesel/diesel blends were diluted with n-heptane in three successive steps. In each step 0.7 ml of the blend was accurately mixed with 9.3 ml of n-heptane. The final dilution comprised 1:2915, or 0.0343%, (v/v) biodiesel/diesel blend in n-heptane. This dilution reduced the absorbance in the 240-350 nm wavelength range to a measurable range for all biodiesel/diesel samples.

### Calculation of Absorbance Index for Biodiesel/Diesel Blends by Ultraviolet Spectroscopy

In one embodiment of the method, biodiesel/diesel blends were formed. The resulting blends were analyzed using UV spectroscopy. Absorbances were measured at 265, 273 and 280 nm. In another embodiment, absorbances were measured at 260, 266 and 280 nm. The values of AI were calculated at various blend levels from B5 to B80 using equation 1 for the absorbances measured at 265, 273 and 280 nm. The calculated AI values for the blends were linearly correlated with the blend level. The coefficients of variation (CV) of AI for diesel fuels were found to be low. The mean and CV of AI are shown in Table 1:

TABLE 1
Mean and coefficient of variation of AI for different biodiesel blends.

| Blend | Mean | CV |
|---|---|---|
| B0 | 1.1135 | 3.70 × 10⁻³ |
| B5 | 1.1055 | 3.48 × 10⁻³ |
| B10 | 1.0976 | 1.23 × 10⁻³ |
| B20 | 1.0864 | 2.66 × 10⁻³ |
| B30 | 1.0766 | 1.69 × 10⁻³ |
| B50 | 1.0530 | 2.86 × 10⁻³ |
| B80 | 1.0226 | 3.45 × 10⁻³ |

The mean AI for all diesels was found to be 1.1135 with CV of 3.70×10⁻³, and for B80, mean AI was 1.0226 with CV of 3.45×10⁻⁴. Even though the absolute difference in mean AI between neat diesel and B80 was small, the very small CV values made the blend level prediction reliable.

### Determination of Relative Amounts of Biodiesel/Diesel in a Blend by Near Ultraviolet Spectroscopy

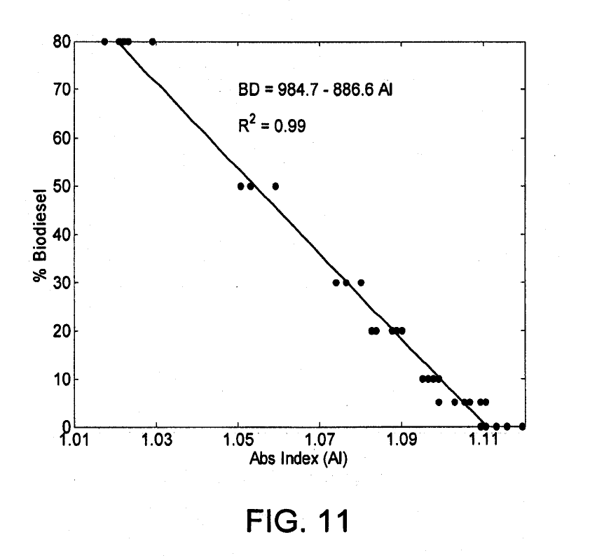

In one embodiment of the method, biodiesel/diesel blends containing 5, 10, 20, 30, 50 and 80% (v/v) biodiesel were prepared and sequentially diluted with n-hexane to a final ratio of 1:2915. An aliquot of the diluted blend was placed into a 1-cm quartz cuvet. The absorbance of each diluted blend was measured at 265, 273 and 280 nm. Several trials were run for each blend. For each trial, the absorption index AI was calculated using Eq. 1 and the resulting values were plotted against the percentage biodiesel as shown in FIG. 11.

A best fit line was determined. The R² value of the fitted line was found to be 0.99. The root mean squared error (RMSE) of the line was 2.88%. The linear equation was:

BD=984.7−886.6 AI (Eq. 4)

where BD is the blend level and AI is the absorbance index from equation 1. It is clear from Eq. 4 that the predicted blend level is very sensitive to AI. However, the coefficient of variation in measuring AI was very small. From Table 1, the maximum observed coefficient of variation was 3.7×10⁻³. This translates to a maximum error in percent biodiesel prediction of 3.28%. In this example, the disclosed method predicted biodiesel percentage with an average accuracy of ±2.88%.

### Determination of Relative Amounts of Biodiesel/Diesel in a Blend by Near Infrared Spectroscopy

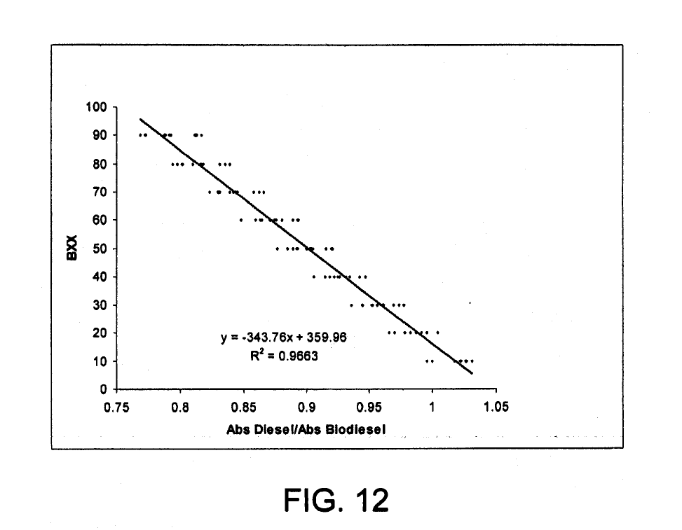

In one embodiment of the method, biodiesel/diesel blends were prepared from different feedstocks. An aliquot of each blend was placed into a 5-cm cuvet. The absorbance of each blend was measured at 917 nm, 929 nm, 970 nm, and 800 nm. For each blend, the ratio of diesel absorbance to biodiesel absorbance was calculated using Eq. 2, and the resulting values were plotted against the blend level as shown in FIG. 12. A best-fit line was determined. The R² value of the fitted line was found to be 0.966. The linear equation was

y=−343.76x=359.96 (Eq. 5)

where y is the blend level and x is the ratio of diesel absorbance to biodiesel absorbance.
Calculation of Blend Level from Detector Output Signal

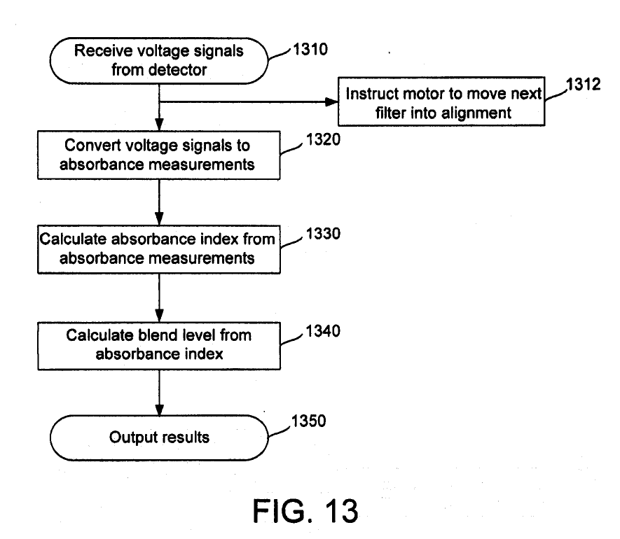

FIG. 13 is a flow chart of the software instructions performed by a data analyzer of the invention in one working embodiment. In step 1310, a voltage signal from the detector was received by the data analyzer for a first wavelength used for a particular blend. Step 1310 was repeated two additional times at the first wavelength. In step 1312, after receiving the voltage signals, the data analyzer instructed the motor to move the next filter into alignment between the light source and the detector. The detector subsequently sent additional voltage signals, corresponding to the next wavelength, to the data analyzer. The process of receiving voltage signals (step 1310), instructing the motor to move (step 1312) and receiving additional voltage signals was repeated for each of the measured wavelengths. Each of the voltage signals received from the detector was converted to an absorbance measurement in step 1320. Using Eq. 1, the absorbance index for the blend was calculated from the absorbance measurements in step 1330. Using Eq. 4, the absorbance index was used to calculate the blend level in step 1340. The results of the blend level calculation were output in step 1350.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method for determining relative amounts of biodiesel and diesel in a biodiesel/diesel blend without separating the biodiesel from the diesel, the method comprising:
providing a device for measuring absorbance of the blend;
measuring absorbance of the blend at one or more wavelengths; and
determining the relative amounts of biodiesel and diesel in the blend from the absorbance.

2. The method of claim 1 where the biodiesel/diesel blend is diluted with a non-aromatic solvent such that the absorbance is less than 2.

3. The method of claim 2 where the blend is diluted with the non-aromatic solvent to a final concentration from about 0.02% (v/v) to about 0.05% (v/v).

4. The method of claim 1 where providing the device comprises having a light source, where the light source produces at least ultraviolet light.

5. The method of claim 1 where the one or more wavelengths is within the range from about 200 nm to about 320 nm.

6. The method of claim 1 where the absorbance of the blend is measured at a first wavelength, a second wavelength and a third wavelength, and where the first, second and third wavelengths are between about 260 nm and about 285 nm.

7. The method of claim 6 where the second wavelength is longer than the first wavelength and the third wavelength is longer than the second wavelength.

8. The method of claim 6 where the first wavelength is about 265 nm, the second wavelength is about 273 nm and the third wavelength is about 280 nm.

9. The method of claim 1 where providing the device comprises having a light source, where the light source produces at least infrared light.

10. The method of claim 1 where the one or more wavelengths is within the range from about 750 nm to about 1100 nm.

11. The method of claim 1 where the device comprises having a light source, where the light source produces at least ultraviolet light and infrared light.

12. The method of claim 1 where the one or more wavelengths is within the range from about 200 nm to 320 nm and/or within the range from about 750 nm to about 1100 nm.

13. The method of claim 1 where determining the relative amounts of biodiesel and diesel in the biodiesel/diesel blend comprises:
preparing a standard curve of percent diesel versus absorbance index for known concentrations of diesel;
determining a best fit straight line of the standard curve; and
determining the relative amount of diesel in the biodiesel/diesel blend utilizing the best fit straight line of the standard curve.

14. A method for making biodiesel, preparing a biodiesel/diesel blend and determining relative amounts of biodiesel and diesel in the biodiesel/diesel blend without separating the blended biodiesel and diesel, the method comprising:
producing biodiesel;
obtaining diesel fuel;
forming a biodiesel/diesel blend;
providing a device for measuring absorbance of the blend;
measuring absorbance of the blend at one or more wavelengths; and
determining the relative amounts of biodiesel and diesel in the blend from the absorbance.

15. A device, comprising:
a light source; and
a detector for detecting light transmitted through a sample comprising a biodiesel/diesel blend.

16. The device of claim 15 where the light source is at least one light-emitting diode of a discrete wavelength.

17. The device of claim 15 where the light source produces at least ultraviolet light.

18. The device of claim 15 where the light source produces at least infrared light.

19. The device of claim 15 where the light source produces at least ultraviolet light and infrared light.

20. The device of claim 15 further comprising at least one filter where only light of a particular wavelength or wavelengths passes through the filter.

21. The device of claim 20 where the at least one filter is located between the light source and the detector.

22. The device of claim 15 further comprising a data analyzer for computing relative amounts of biodiesel and diesel in the biodiesel/diesel blend.

23. The device of claim 22 where the detector outputs a signal, and the data analyzer contains instructions for performing data analysis of the signal.

24. A device, comprising:
a light source;
a sample holder;
a first filter, a second filter and a third filter effectively coupled to a filter holder;
a motor coupled to the filter holder for serially positioning each filter in a light path produced by the light source;
a detector for detecting light transmitted through a sample comprising a biodiesel/diesel blend; and
a data analyzer for computing relative amounts of biodiesel and diesel in the biodiesel/diesel blend.

25. The device of claim 24 where light of about 260 nm passes through the first filter, light of about 266 nm passes through the second filter, and light of about 280 nm passes through the third filter.
